(19) United States
(12) Patent Application Publication
(10) Pub. No.: US 2015/0144143 A1
(43) Pub. Date: May 28, 2015

(54) Pressure sore treatment device and method

(71) Applicant: Sandra J. Forsythe, Eatonville, WA (US)

(72) Inventor: Sandra J. Forsythe, Eatonville, WA (US)

(21) Appl. No.: 14/088,431

(22) Filed: Nov. 24, 2013

(51) Int. Cl. A61F 5/32

(52) U.S. Cl. CPC A61F 5/32
USPC 128/888

(57) ABSTRACT

A pressure sore treatment device for reducing pressure on an area of the skin of a patient, while ensuring air circulation to the skin, can include: a cover box, which can further include a box structure with a hole; and a belt strap, so that the cover box can be placed with the hole covering the affected area of the skin, and secured to the body of the patient with the belt strap. The cover box can be detachable from the belt strap, and in some versions the device can be made from antimicrobial materials. Further disclosed is a method for pressure sore treatment including: placing a box structure with a hole over a treatment area on a patient's body and securing the box structure in place over the treatment area, so that the hole covers and eliminates pressure on the treatment area.

### CROSS-REFERENCE TO RELATED APPLICATIONS

N/A

### FIELD OF THE INVENTION

The present invention relates generally to the field of devices and methods for treating pressure sores.

### BACKGROUND OF THE INVENTION

A pressure sore, also referred to as a pressure ulcer, decubitus ulcer, or bedsore, is a localized breakdown of skin caused by constant pressure on one location. Generally, treatment proceeds by reduction of pressure in the affected area, and by ensuring air circulation.

It is well established that pressure sores can be very difficult to prevent or treat in older bed confined or critically ill patients, or individuals who are wheel chair users.

Well-known prevention methods are based on redistributing pressure by turning or repositioning the patient regularly. Special bed devices and mattress designs have been employed to facilitate easy rotation or altered pressure distribution. However, these devices have often proven expensive and cumbersome in use, and are not always effective in alleviating pressure in all locations on the human body.

As such, considering the foregoing, it may be appreciated that there continues to be a need for novel and improved devices and methods to treat and prevent pressure sores.

### SUMMARY OF THE INVENTION

The foregoing needs are met, to a great extent, by the present invention, wherein in aspects of this invention, enhancements are provided to the existing models of pressure sore prevention and treatment.

In an aspect of the present invention, a pressure sore treatment device for reducing pressure on an area of the skin of a patient, and thereby treating or preventing a pressure sore, can include a cover box, which further contains a cover box cavity, which goes through the cover box, with openings on the outer and inner sides of the cover box, so that the cover box can be placed over the area of the skin of the patient, whereby the opening on the inner side of the box structure reduces pressure on the affected area of the skin.

In a related aspect, the pressure treatment device can further include a belt strap to secure the pressure sore treatment device to an area of a patient's body.

In a related aspect, the cover box and the belt strap can further include a fastening mechanism so that the cover box can be attached to and removed from the belt strap.

In a related aspect, the cover box can be made with an inner core of a suitably firm foam material, which furthermore can be covered by an external cover made from a soft medical textile.

In a further related aspect, both the belt strap and the external cover can be made from an antimicrobial medical textile material, such as for example an antiseptic bio-textile.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a view of the inner side of the belt strap, according to an embodiment of the invention.

FIG. 2 is a perspective view of the inner side of the cover box, according to an embodiment of the invention.

FIG. 3 is a perspective view of the outer side of the cover box, according to an embodiment of the invention.

FIG. 4 is a perspective view of the inner side of the pressure sore treatment device, according to an embodiment of the invention.

FIG. 5 is a perspective view of the outer side of the pressure sore treatment device, according to an embodiment of the invention.

FIG. 6 is a perspective view illustrating how the pressure sore treatment device can be mounted on a body part of a patient, according to an embodiment of the invention.

### DETAILED DESCRIPTION

Before describing the invention in detail, it should be observed that the present invention resides primarily in a novel and non-obvious combination of elements and process steps. So as not to obscure the disclosure with details that will readily be apparent to those skilled in the art, certain conventional elements and steps have been presented with lesser detail, while the drawings and specification describe in greater detail other elements and steps pertinent to understanding the invention.

The following embodiments are not intended to define limits as to the structure or method of the invention, but only to provide exemplary constructions. The embodiments are permissive rather than mandatory and illustrative rather than exhaustive.

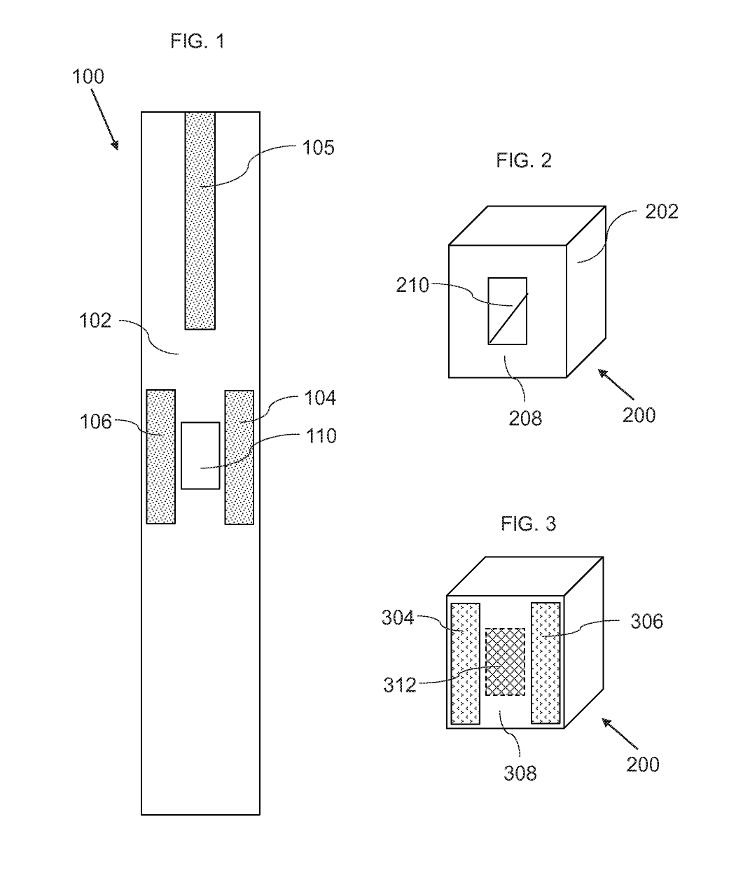

In the following we describe the structure of such an embodiment in the form of a pressure sore treatment device 400 with reference to FIGS. 1, 2, 3, 4, and 5, in such manner that like reference numerals refer to like components throughout; a convention that we shall employ for the remainder of this specification.

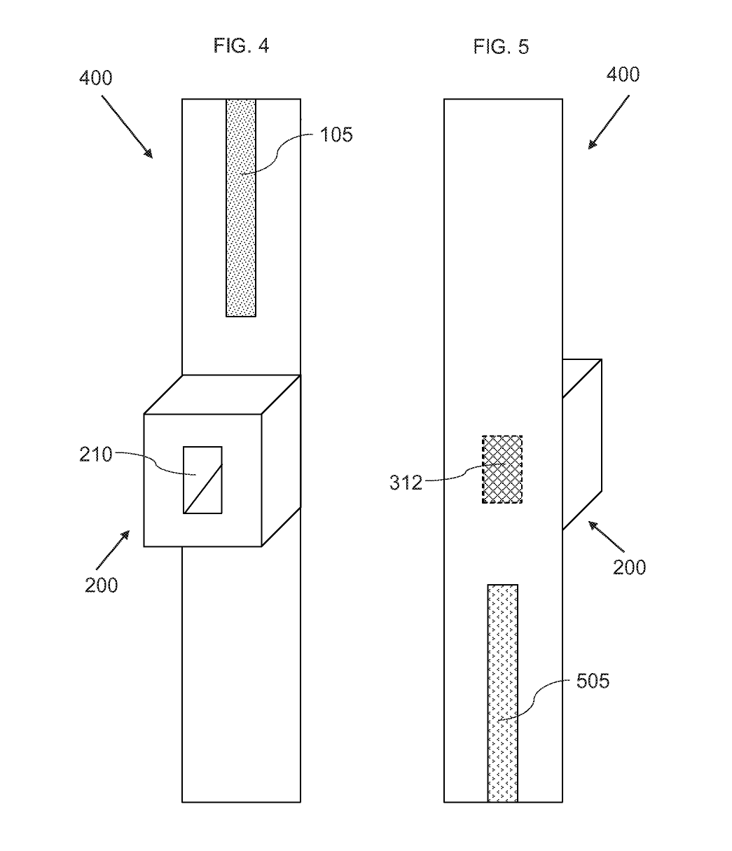

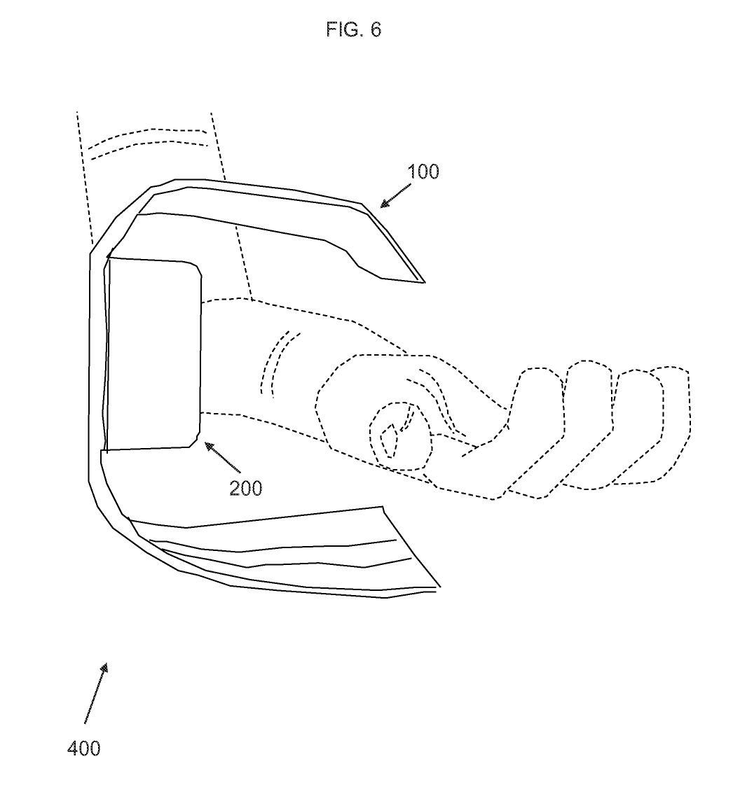

In an embodiment, illustrated in FIGS. 4 and 5, a pressure sore treatment device 400 for reducing pressure on an area of the skin of a patient, while ensuring air circulation to the area of the skin, can be comprised of:
a. A cover box 200, illustrated in FIGS. 2 and 3, which can further comprise:
   i. a box structure 202, which further includes a
      1. a box structure cavity 210, which forms a hole going through the box structure 202, with openings on respectively the outer side 308, and the inner side 208;
   ii. a box-belt fastening mechanism, which can further include:
      1. A left box fastening pad 304, attached to the outer side 308, of the box structure 202, to the left of the hole opening on the outer side 308 and
      2. A right box fastening pad 306, attached to the outer side 308, of the box structure 202, to the right of the hole opening on the outer side 308;
b. A belt strap 100, illustrated in FIG. 1, comprised of
   i. A belt body 102, which further comprises a belt body hole 110, of similar size and shape to the size and shape of the opening of the box structure cavity 210, on the surface of the outer side 308, wherein the belt body can be made of a flexible medical fabric material;
   ii. A belt-to-box fastening mechanism, which can further include:
      1. A left belt fastening pad 104, attached to the inner surface of the belt body 102, on the left side of the belt body hole 110;
      2. A right belt fastening pad 106, attached to the inner surface of the belt body 102, on the right side of the belt body hole
   iii. A belt-to-belt fastening mechanism, illustrated in FIGS. 4 and 5, which can further include:
      1. An outer fastening pad 105, of a rectangular shape, positioned on the inner surface of a first end of the belt body 102; and
      2. An inner fastening pad 505, of a rectangular shape, positioned on the outer surface of a second end of the belt body 102;
   Wherein the right and left belt fastening pads 104 106, can stick to respectively the right and left box fastening pads 304 306, and as illustrated in FIG. 6, the pressure sore cover 400, can be mounted on a pressure sore, and secured by tightening the belt strap 100, and securing the belt strap in place by sticking together the outer fastening pad 104 with the inner fastening pad 504.

In an embodiment, the cover box 200 can be used without the belt strap 100, for example by positioning underneath a patient lying in a bed. It can for example be used alleviate pressure on the tailbone of a patient, who is lying on the back, so that the cover box 200 is placed under the patient, with the opening on the inner side 208 placed directly under the tailbone, thereby alleviating pressure on the tailbone area and reducing the risk of a bedsore in this area.

In various related embodiments, the box-to-belt, belt-to-box, and belt-to-belt fastening mechanisms can use a variety of fastening mechanisms, in a variety of configurations and shapes, such as for example:
a. hook-and-loop fasteners, commonly referred to as velcro fasteners, in which case for example:
   i. the left belt fastening pad 104, the right belt fastening pad 106, and the outer fastening pad 105, can each be made of either a hook or a loop material, and respectively the left box fastening pad 304, the right box fastening pad 306, and the inner fastening pad 505, can each be made of e a corresponding loop or hook material; or
   ii. in another example, a different number of hook-and-loop fastener pads, further employing different shapes of pads, can be used to ensure the box can be attached to the belt, and the belt can be secured firmly around a body part.
b. stick-and-peel fasteners, employing a weak glue or sticky material;
c. buttons;
d. snap-lock buttons; and
e. laces, for tying together;

In a related embodiment, a mesh fabric cover 312 can cover the opening on the outer side 308 of the box structure cavity 210, and thereby allow air circulation, while preventing foreign objects to enter the box structure cavity 210

In a related embodiment, the pressure sore cover 400 can be permanently attached to the belt strap 100, for example by being sewn or glued together.

In related embodiments, the left and right belt fastening pads, and the inner and outer fastening pads 104 504, can be attached to the belt body 102, by common fastening methods, such as sewn stitches or glue.

In a related embodiment, the box structure 202 can be made of a firm foam material, such as for example an extruded closed-cell polystyrene foam in a density range from 25 to 50 kg/m³. Alternatively, it can be another type of firm foam, such as for example an open-cell flexible polyurethane foam with a density in a range from 40 to 80 kg/m³. In general, the foam material can be an expanded or extruded, open- or closed-cell foam. In these various embodiments, the foam material should have a sufficient firmness that is comfortable but does not exhibit excessive deformation in normal use, which could deform the hole openings of the box structure 202, and thereby reduce the effectiveness of the pressure sore treatment device 400. A foam of sufficient firmness can have an indentation force deflection at 25% of 40-80 pounds/50 in², but this can vary depending on application and type of foam.

In a further related embodiment, the box structure 202 can be made of a reticulated foam material, of sufficient firmness, whereby the box structure can allow for excess moisture or liquid to evaporate or drain off, and further can allow for easy cleaning. A reticulated foam of sufficient firmness can for example be a reticulated polyurethane foam with a density in a range of 20 to 50 kg/m³.

In a related embodiment, the box structure 202 can further include an external cover made by a medical fabric material, such as a soft medical cotton material.

In further related embodiments, parts of or all of the pressure sore cover device 400, can be antimicrobial, for example via treatment with an antimicrobial agent, or via construction from materials that are inherently antimicrobial. This can include treatment with or use of various well-known agents or materials with antibiotic and/or antifungal properties.

In a further related example embodiment, both the belt body 102 and the external cover of the box structure 202, can be made of a medical fabric, which is an antiseptic bio-textile, for example a medical fabric, composed of a cotton textile treated with Zeolite or Triclosan.

In a related embodiment, the belt body 102 can be made of an elastic medical fabric.

In various related embodiments, the pressure sore treatment device 400 can be made in a plurality of sizes and specific shapes to fit different parts of the human body. In an example embodiment, suitable for general use, the box structure can have a depth, equal to the depth of the hole from the outer side 308 to the inner side 208, of 2 inches, and an equal length and width of 4 inches, whereby the outer side 308 and the inner side 208 are quadratic, with side lengths of 4 inches. In this example embodiment, the openings of the hole on respectively the outer side 308 and the inner side 208 can be centrally located on each outer and inner side 308 208 of the box structure, and be quadratic with a length and width of 1.5 inches, and a depth of 2 inches, similar to the depth of the box structure. The belt strap 100, can in this example embodiment have a length of 23 inches, and width, similar to the width of the box structure, of 4 inches.

A method for preventing or treating pressure sores can comprise:
a. Placing a box structure with a hole over a treatment area on a humans body, so that the hole covers and eliminates pressure on the treatment area;
b. Securing the box structure in place over the treatment area, by tightening and fastening a belt attached to the box structure.

In a further embodiment, the method can further comprise: applying a protective skin ointment, such as petroleum jelly, or similar skin ointment, to the treatment area, before placing the box structure over the treatment area.

In a limited study of an embodiment of the pressure sore treatment device 400, a patient had suffered a bone fracture in the upper leg, and had to have pins embedded in the leg to keep bones in place during healing. Pressure from the other leg, caused the pins to rupture the skin, resulting in significant pain, and a progressively worsening sore, that did not respond to standard attempts at treatment. After installation of an embodiment of the pressure sore treatment device 400, the patient's condition improved, the skin healed, and pain subsided. In continuing preventive treatment, there were no further episodes of skin breaking.

The many features and advantages of the invention are apparent from the detailed specification, and thus, it is intended by the appended claims to cover all such features and advantages of the invention, which fall within the true spirit and scope of the invention.

Many such alternative configurations are readily apparent, and should be considered to be fully included in this specification and the claims appended hereto. Accordingly, since numerous modifications and variations will readily occur to those skilled in the art, it is not desired to limit the invention to the exact construction and operation illustrated and described, and thus, all suitable modifications and equivalents may be resorted to, falling within the scope of the invention.

What is claimed is:

1. A pressure sore treatment device for reducing pressure on an area of the skin of a patient, and thereby treating or preventing a pressure sore, comprising:
a cover box, which further comprises a box structure with a box structure cavity, wherein further the box structure cavity forms a hole going through the box structure, with openings on respectively an outer side, and an inner side of the box structure;
whereby the cover box can be positioned over the area of the skin of the patient, so that the opening on the inner side of the box structure reduces pressure on the area of the skin.

2. The pressure sore treatment device of claim 1, further comprising:
a belt strap, which further comprises:
a belt-to-belt fastening mechanism; and
a belt body;
wherein the belt strap is attached to the outer side of the cover box without covering the opening to the box structure cavity and the belt strap can be fastened, using the belt-to-belt fastening mechanism, around a part of the body of the patient; whereby the cover box can be secured in place over the area of the skin of the patient, so that the opening on the inner side of the box structure reduces pressure on the area of the skin.

3. The pressure sore treatment device of claim 2, wherein
a) the cover box further comprises a box-belt fastening mechanism; and
b) the belt strap further comprises a belt-box fastening mechanism;
wherein the cover box can be securely attached to the belt strap, and is removable.

4. The pressure sore treatment device of claim 2, wherein the cover box and the belt strap are permanently attached.

5. The pressure sore treatment device of claim 2,
wherein a left belt fastening pad, a right belt fastening pad, and an outer fastening pad, are each made of either a hook or a loop material, and respectively a left box fastening pad, a right box fastening pad, and an inner fastening pad, are each made of a corresponding loop or hook material;
whereby the box can be attached to the belt, and the pressure sore treatment device can be secured firmly around a body part, and whereby further after the removal of the pressure sore treatment device from the body part, the cover box can be detached from the belt strap.

6. The pressure sore treatment device of claim 1, wherein the opening on the outer side of the box structure is covered by a mesh fabric cover, whereby the mesh fabric cover can allow air circulation, while preventing foreign objects to enter the box structure cavity.

7. The pressure sore treatment device of claim 1, wherein the box structure is made of a foam material of sufficient firmness.

8. The pressure sore treatment device of claim 7, wherein the foam material is a reticulated foam.

9. The pressure sore treatment device of claim 8, wherein the foam material is a reticulated polyurethane foam with a density in a range from 20 to 50 kg/m³.

10. The pressure sore treatment device of claim 7, wherein the foam material is antimicrobial.

11. The pressure sore treatment device of claim 1, wherein the box structure further comprises an external cover made by a medical textile material.

12. The pressure sore treatment device of claim 11, wherein the external cover of the box structure is made from an antiseptic bio-textile.

13. The pressure sore treatment device of claim 2, wherein the belt body of the belt strap is made from an antiseptic bio-textile.

14. The pressure sore treatment device of claim 2, wherein the belt body of the belt strap is made of an elastic medical fabric.

15. A method for preventing or treating pressure sores can comprise:
b) placing a cover box, which is configured with a hole that penetrates the cover box from an outer side to an inner side, over a treatment area on a human's body, so that the hole on the inner side of the cover box covers the treatment area, whereby pressure on the treatment area is eliminated or reduced.

16. The method for preventing or treating pressure sores of claim 15, further comprising:
c) securing the cover box in place over the treatment area, by tightening and fastening a belt attached to the cover box.

17. The method for preventing or treating pressure sores of claim 15, further comprising:
a) applying a protective skin ointment to the treatment area, before placing the box structure over the treatment area.

18. The method for preventing or treating pressure sores of claim 15, wherein the hole on the outer side of the cover box is further covered with a mesh fabric.

19. The method for preventing or treating pressure sores of claim 15, wherein the cover box is configured with an inner core made of a foam material that is a reticulated polyurethane foam with a density in a range from 20 to 50 kg/m³.

20. The method for preventing or treating pressure sores of claim 16, wherein all surfaces of the belt and the box structure are constructed from an antiseptic bio-textile.
